(19) United States
(12) Patent Application Publication
(10) Pub. No.: US 2025/0026916 A1
(43) Pub. Date: Jan. 23, 2025

(54) ULTRAVIOLET ABSORBER AND USE THEREOF

(71) Applicant: RIANLON (ZHONGWEI) NEW MATERIAL CO., LTD., ZHONGWEI CITY (CN)

(72) Inventors: Wei YANG, Tianjin (CN); Donghui SUN, Tianjin (CN); Yanqing LI, Tianjin (CN); Pengbo ZHAO, Tianjin (CN); Hai LUO, Tianjin (CN); Cai LI, Tianjin (CN)

(21) Appl. No.: 18/762,884

(22) Filed: Jul. 3, 2024

(30) Foreign Application Priority Data
Jul. 4, 2023 (CN) 202310814496.5

(51) Int. Cl. C08K 5/3475; C07D 249/20; C08J 5/06; C09D 5/44; C09D 7/48; G02B 1/04; G02B 5/20

(52) U.S. Cl. CPC C08K 5/3475; C07D 249/20; C08J 5/06; C09D 5/448; C09D 7/48; G02B 1/04; G02B 5/208; C08J 2363/00; C08J 2433/14

(57) ABSTRACT

The present invention in certain embodiments relates to an ultraviolet absorber having a structure represented by formula (I) or formula (II) as disclosed herein and compositions, polymer materials, articles of manufacture, methods and uses thereof.

### TECHNICAL FIELD

The present invention belongs to the field of light stabilizers, and specifically: relates to ultraviolet absorbers.

### BACKGROUND

Polymer materials are indispensable basic materials in many facets of life and society, and the issue of time stability of the polymer materials inevitably affects their usability, causing shortened service life and a waste of resources. The factors causing stability issues over time of polymers are mainly light and heat, among which ultraviolet rays in natural sunlight have a great influence on the long-acting performance of the polymers. At present, the prevention of outdoor photoaging is mainly solved by adding light stabilizers. Such light stabilizers include ultraviolet absorbers and hindered amine light stabilizers. With the gradual penetration and extension of polymer materials from general-purpose and basic materials to special functional materials, some special application scenarios have emerged in the anti-aging of polymer materials, and traditional light stabilizers can no longer fully meet the needs.

Although traditional ultraviolet absorbers have a relatively full coverage in most of ultraviolet regions (wavelength 280-400 nm), the absorption efficiency at wavelengths above 380 nm is significantly reduced. When ultraviolet absorbers are applied to application fields with special requirements in the aspect of a long wave (redshift region), the traditional ultraviolet absorbers are not as useful to meet the desired requirements.

Carbon fibers are an emerging material of great interest. At present, carbon fiber reinforced epoxy resin composite materials are the most widely used carbon fiber reinforced materials (CFRMs) having the highest comprehensive indexes such as strength, modulus, etc. However, epoxy resins are themselves susceptible to damage by ultraviolet rays and visible light below 420 nm, causing degradation, and thus affecting their performance. Ultraviolet absorbers with high absorption efficiency at 380-420 nm can effectively protect such materials.

Polyethylene naphthalate (PEN) is a polymer material with excellent comprehensive properties such as chemical stability, mechanical properties, heat resistance, etc., and has relatively good application prospects in the fields of optical elements and the like. However, ultraviolet rays at 360-400 nm are prone to causing damage to PEN and composite materials containing PEN, resulting in material degradation.

In addition, there are some fields that require special shielding requirements for light in the wavelength range of 360-430 nm. For example, special polarizers are required to absorb ultraviolet rays in the wavelength range of 380 nm and below as completely as possible or at least 75-85% of the ultraviolet rays, while transmitting ultraviolet rays above 410 nm as much as possible, for example, transmitting at least greater than 95% of the ultraviolet rays. Still other glass coatings and films, such as those used in automotive front windshields and special architectural glasses, are required to shield ultraviolet rays below 400 nm as much as possible to meet the higher health requirements of people exposed to such environments. In order to meet such requirements, the ultraviolet absorbers must be red shifted to a higher wavelength, so as to meet the requirement of better absorption efficiency in the of 380-410 nm region. For another example, optical lenses, display screen devices, etc. are required to have relatively high absorption for high-energy blue light in the waveband of 400 nm-445 nm, thereby protecting the vision health of users of electronic products.

In this field, there is an urgent need for an ultraviolet absorber that not only has a good absorption effect on most of ultraviolet regions (280-400 nm), but also keeps a good absorption effect on light with a wavelength above 380 nm, particularly light in a redshifted spectral region.

### SUMMARY

According to the present invention, thioether or sulfonyl is introduced into the conventional benzotriazole absorber, so that the cut-off wavelength of the conventional benzotriazole absorber is red-shifted from 400-410 nm to a long-wave direction up to 430 nm. In addition, by selecting a proper alkyl chain segment, the compound with the novel structure is ensured to have proper melting point and solubility, as well as better system compatibility, making it applicable to a variety of material systems.

One aspect of the present invention provides a compound having a structure represented by formula (I) or formula (II),

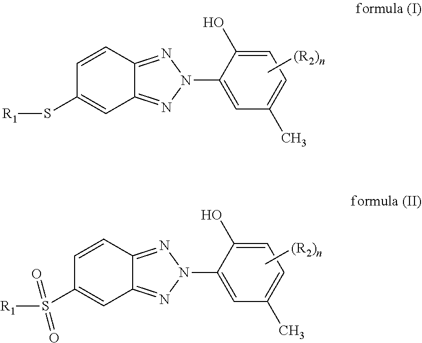

wherein in formula (I) and formula (II), R₁ is selected from C1-C20 linear or branched alkyl, R₂ is selected from C1-C8 linear or branched alkyl, and n is selected from an integer of 1-3.

In some embodiments, in formula (I) and formula (II), R₁ is selected from methyl, ethyl, n-propyl, isopropyl, n-butyl, isobutyl, tert-butyl, pentyl, hexyl, n-heptyl, n-octyl, n-nonyl, n-decyl, n-undecyl, n-dodecyl, n-tridecyl, n-tetradecyl, n-pentadecyl, and the like. In some embodiments, in formula (I) and formula (II), R₁ is selected from C5-C20 linear or branched alkyl, preferably C7-C15 linear or branched alkyl.

In some embodiments, in formula (I) and formula (II), R₂ is selected from methyl, ethyl, propyl, isopropyl, n-butyl, isobutyl, sec-butyl, tert-butyl, 1,1-dimethyl-propyl, and the like. In some embodiments, in formula (II), R₂ is selected from C2-C6 linear or branched alkyl.

In some embodiments, the compound has a structure represented by formula (III) or formula (IV),

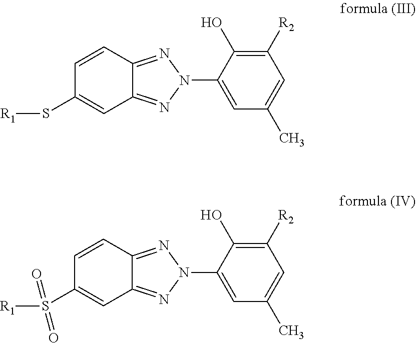

wherein in formula (III) and formula (IV), R₁ is selected from C1-C20 linear or branched alkyl, and R₂ is selected from C1-C8 linear or branched alkyl.

In some embodiments, in formula (III) and formula (IV), R₁ is selected from methyl, ethyl, n-propyl, isopropyl, n-butyl, isobutyl, tert-butyl, pentyl, hexyl, n-heptyl, n-octyl, n-nonyl, n-decyl, n-undecyl, n-dodecyl, n-tridecyl, n-tetradecyl, n-pentadecyl, and the like. In some embodiments, in formula (III) and formula (IV), R₁ is selected from C5-C20 linear or branched alkyl, preferably C7-C15 linear or branched alkyl.

In some embodiments, in formula (III) and formula (IV), R₂ is selected from C2-C6 linear or branched alkyl. In some specific embodiments, R₂ is selected from methyl, ethyl, propyl, isopropyl, n-butyl, isobutyl, sec-butyl, tert-butyl, 1,1-dimethyl-propyl, and the like.

In some embodiments, exemplary compounds of formula (III) include:

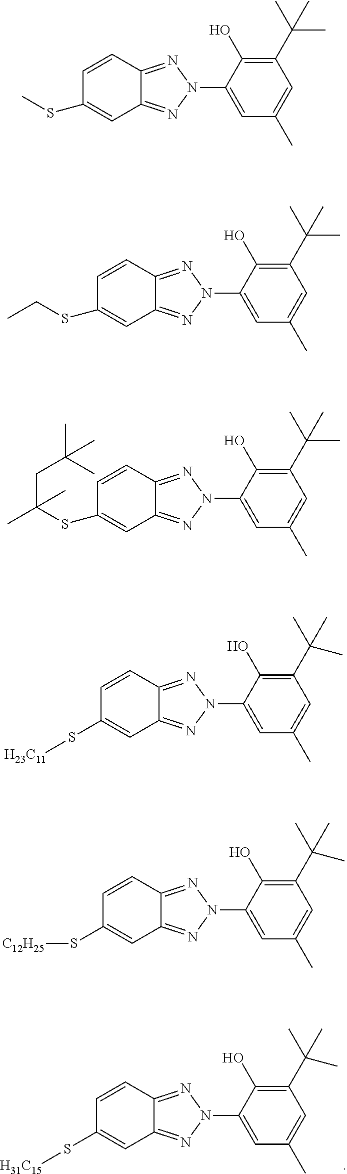

In some embodiments, exemplary compounds of formula (IV) include:

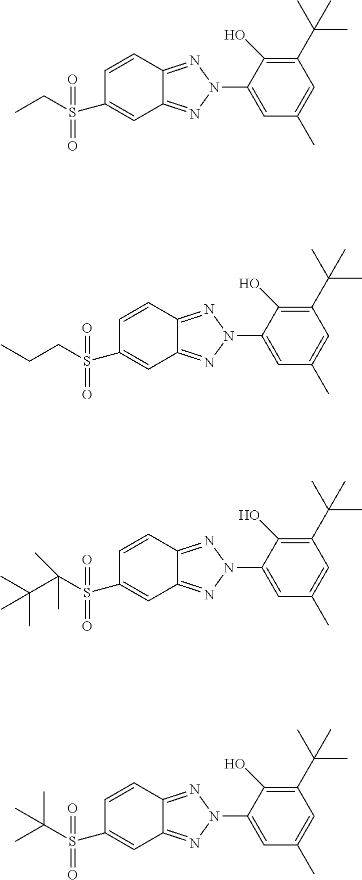

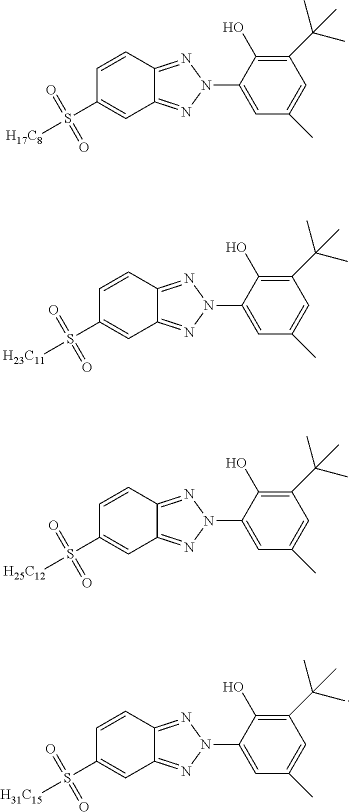

In some preferred embodiments, the compound has a structure represented by RSUV-1 or RSUV-2:

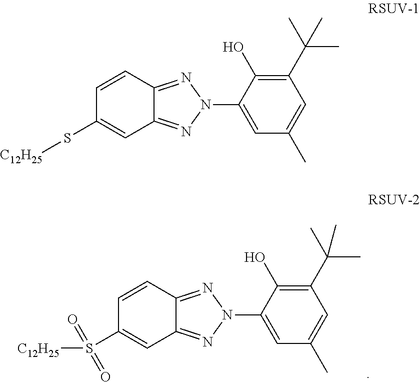

In some embodiments, the compound has an absorption wavelength ranging from 280 nm to 430 nm.

When the compound, particularly the compound of RSUV-1 or RSUV-1, of the present invention is used as an ultraviolet absorber, compared with the traditional ultraviolet absorbers, particularly compared with the conventional benzotriazole ultraviolet absorbers, the absorption for the ultraviolet region can be shifted overall to the long-wave direction, wherein the absorption cut-off wavelength of RSUV-2 can even be red-shifted to 430 nm, meeting the absorption requirements of polymer materials at 280-430 nm. In addition, the compound of the present invention also has a proper melting point, good solubility, better oligomer resin system compatibility, lighter chroma, and excellent comprehensive properties.

Another aspect of the present invention provides a method for preparing the compound described above, the method comprising: using a compound having a structure represented by formula (V) as a raw material and reacting it with alkyl thiol to obtain the compound having the structure represented by formula (I).

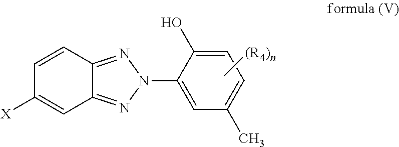

wherein,
in formula (V), X is selected from halogen, such as F, Cl, Br, or I, preferably Cl, R₄ is selected from C1-C8 linear or branched alkyl, preferably C2-C6 linear or branched alkyl, and n is selected from an integer of 1-3.

In some embodiments, the alkyl thiol has a structure represented by HS—R₃, wherein R₃ is selected from C1-C20 linear or branched alkyl, preferably C5-C20 linear or branched alkyl, and more preferably C7-C15 linear or branched alkyl.

In some embodiments, the preparation method further comprises oxidizing the compound having the structure represented by formula (I) by an oxidant to obtain the compound having the structure represented by formula (II).

In some embodiments, the oxidant may be an oxidant, such as hydrogen peroxide.

In some embodiments, the preparation method comprises using a compound having a structure represented by formula (VI) as a raw material and reacting it with alkyl thiol to obtain the compound having the structure represented by formula (III),

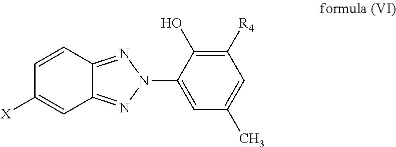

wherein in formula (VI), X is selected from halogen, such as F, Cl, Br, or I, preferably Cl, and R₄ is selected from C1-C8 linear or branched alkyl, preferably C2-C6 linear or branched alkyl.

In some embodiments, the preparation method further comprises oxidizing the compound having the structure represented by formula (III) by an oxidant to obtain the compound having the structure represented by formula (IV).

Yet another aspect of the present invention provides a composition comprising the compound described above.

In some embodiments, the composition further comprises one or more additives.

In some embodiments, the additive includes, but is not limited to, one or more of a hindered amine light stabilizer, an additional ultraviolet absorber, an antioxidant, an emulsifier, a nucleating agent, a toughening agent, a lubricant, an anti-blocking agent, a filler, a dye, a pigment, a fluorescent brightener, a flame retardant, an antistatic agent, a foaming agent, or the like.

In some embodiments, the hindered amine light stabilizer is a hydroxy substituted alkoxyamine stabilizer.

In some embodiments, the hindered amine light stabilizer includes, but is not limited to: 1-cyclohexyloxy-2,2,6,6-tetramethyl-4-octadecylaminopiperidine; 1,2,2,6,6-pentamethyl-4-aminopiperidine; bis(1-acyl-2,2,6,6-tetramethylpiperidin-4-yl)sebacate; bis(1-octyloxy-2,2,6,6-tetramethylpiperidin-4-yl)sebacate; bis(1,2,2,6,6-pentamethyl-4-yl)sebacate; mono(1,2,2,6,6-pentamethyl-4-yl)sebacate; linear or cyclic condensates of N,N′-bis(2,2,6,6-tetramethyl-4-piperidinyl)-hexamethylenediamine and 4-tert-octylamino-2,6-dichloro-1,3,5-triazine (UV-944); ethyl 2-[2,2,6,6-tetramethyl-4-(3,5,5-trimethyl-hexanoyloxy)-piperidinyl]-3,5,5-trimethylhexanoate; [[3,5-di-tert-butyl-4-hydroxyphenyl]methyl]butyl-di-(1,2,2,6,6-pentamethyl-4-yl)malonate; poly[1-(2′-hydroxyethyl)-2.2.6.6-tetramethyl-4-hydroxypiperidine succinate]; and 1,5,8,12-tetrakis[2,4-bis(N-butyl-N-1,2,2,6,6-pentamethyl-4-piperidinylamino)-1,3,5-triazin-6-yl]-1,5,8,12-tetraazadodecane.

In some embodiments, the additional UV absorber includes benzophenone ultraviolet absorbers, salicylate ultraviolet absorbers, benzotriazole ultraviolet absorbers, substituted acrylonitrile ultraviolet absorbers, triazine ultraviolet absorbers, oxanilide ultraviolet absorbers, cyanoacrylate ultraviolet absorbers, and the like.

In some embodiments, the benzotriazole ultraviolet absorbers include, but are not limited to, 2-(2-hydroxy-5-methylphenyl)-2H-benzotriazole, 2-(2-hydroxy-5-tert-octyl)-2H-benzotriazole, 2-(2-hydroxy-3-cumyl-5-tert-octylphenyl)-2H-benzotriazole, 5-chloro-2-(3,5-di-tert-butyl-2-hydroxyphenyl)-2H-benzotriazole, 5-chloro-2-(3-tert-butyl-2-hydroxy-5-methylphenyl)-2H-benzotriazole, 2-(2-hydroxy-4-octyloxyphenyl)-2H-benzotriazole, 2-(3,5-di-tert-pentyl-2-hydroxyphenyl)-2H-benzotriazole, 2-(3-dodecyl-2-hydroxy-5-methylphenyl)-2H-benzotriazole, 2-(3-tert-butyl-2-hydroxy-5-(2-octyloxycarbonyl)ethylphenyl)-2H-benzotriazole, dodecylated 2-(2-hydroxy-5-methylphenyl)-2H-benzotriazole, 2-(3-tert-butyl-2-hydroxy-5-(2-octyloxycarbonylethyl)phenyl)-5-chloro-2H-benzotriazole, 2-(3-tert-butyl-5-(2-(2-ethylhexyloxy)-carbonylethyl)-2-hydroxyphenyl)-5-chloro-2H-benzotriazole, 2-(3-tert-butyl-2-hydroxy-5-(2-methoxycarbonylethyl)phenyl)-5-chloro-2H-benzotriazole, 2-(3-tert-butyl-2-hydroxy-5-(2-methoxycarbonylethyl)phenyl)-2H-benzotriazole, 2-(3-tert-butyl-5-(2-(2-ethylhexyloxy)carbonylethyl)-2-hydroxyphenyl)-2H-benzotriazole, 2-(3-tert-butyl-2-hydroxy-5-(2-isooctyloxycarbonylethyl)phenyl-2H-benzotriazole, 2,2′-methylene-bis(4-tert-octyl-(6-2H-benzotriazol-2-yl)phenol), 2-(2-hydroxy-3-α-isopropylphenyl-5-tert-octylphenyl)-2H-benzotriazole, and 2-(2-hydroxy-3-tert-octyl-5-α-isopropylphenylphenyl)-2H-benzotriazole.

In some embodiments, the antioxidant includes phenolic antioxidants, amine antioxidants, phosphite antioxidants, thioester antioxidants, and benzofuranone antioxidants.

In some embodiments, the mass percentage of the compound in the composition is 0.1-6%, e.g., about 0.1%, about 0.5%, about 1%, about 1.5%, about 2%, about 3%, about 4%, about 4.5%, about 5%, about 5.5%, about 6%, or any value therebetween. In other embodiments, the mass percentage of the compound in the composition is from any one of about 0.1%, about 0.2%, about 0.5%, about 1%, about 1.5%, or about 2% to any one of about 3%, about 4%, about 4.5%, about 5%, about 5.5%, or about 6%.

In some embodiments, the composition is present in one or more of: an epoxy resin-based article of manufacture and composite articles of manufacture thereof (e.g., epoxy-reinforced carbon fiber composite materials), a polyethylene naphthalate-based article of manufacture, an optical adhesive, an optical element, an optical film (e.g., an optical film taking cellulose triacetate, polycarbonate, polyacrylate, or polyethylene naphthalate as a substrate), an optical lens, an anti-blue light article of manufacture (e.g., an anti-blue light lens, an anti-blue light display device, etc.), a vehicle window and a film thereof, a vehicle paint protection film, an automotive paint (i.e., an automotive coating material, such as a cathodic electrodeposition primer and a coating compounded therewith), and an adhesive.

In some embodiments, the composition is present in a laminated or multilayer structure.

In some embodiments, the composition is a laminated material or a multilayer material. In some embodiments, the laminated material or multilayer material may be a reflective sheet and a conformable marking sheet, a solar control film, a solar reflector, a reflective printed label, a UV absorbing glass and a glass coating material, an electrochromic device, a film/a glazing, a windshield, a middle layer, and the like.

Yet another aspect of the present invention provides a polymer material comprising the compound or the composition described above, and an organic material susceptible to degradation induced by oxygen, heat, or light.

In some embodiments, the organic material in the polymer material includes, but is not limited to, polyester, polyurethane, polyacrylic acid, polycarbonate, epoxy and modified resins thereof, phenolic resin, polyamide, polyimide, polystyrene and derivatives thereof, polysilane, polysiloxane and modifications thereof, poly(vinyl butyral), amino acrylic acid, hydroxyl acrylic acid, polyurethane acrylate, polycyanoacrylate, polyacrylate, ethylene/acrylic acid copolymers and salts thereof (ionomers), poly(vinyl alcohol), cellulose triacetate, polycarbonate, polyethylene naphthalate, polyethylene terephthalate, polyvinyl alcohol, polymethyl methacrylate, polycyclopentene, aliphatic isocyanate, polymethyl methacrylate, fluorine/silicon-modified polyacrylic acid, and polyisocyanate/polythioether.

In some embodiments, the mass percentage of the compound in the polymer material is 0.1-6%, e.g., about 0.1%, about 0.5%, about 1%, about 1.5%, about 2%, about 3%, about 4%, about 4.5%, about 5%, about 5.5%, about 6%, or any value therebetween. In other embodiments, the mass percentage of the compound in the polymer material is from any one of about 0.1%, about 0.2%, about 0.5%, about 1%, about 1.5%, or about 2% to any one of about 3%, about 4%, about 4.5%, about 5%, about 5.5%, or about 6%.

Yet another aspect of the present invention further provides an article of manufacture comprising the compound described above, the composition described above, or the polymer material described above.

In some embodiments, the article of manufacture includes, but is not limited to: an epoxy resin-based article of manufacture and composite articles of manufacture thereof, a polyethylene naphthalate-based article of manufacture, an optical adhesive, an optical element, an optical film (e.g., an optical film taking cellulose triacetate, polycarbonate, polyacrylate, or polyethylene naphthalate as a substrate), an optical lens, an anti-blue light article of manufacture (e.g., an anti-blue light lens, an anti-blue light display device, etc.), a reflective sheet and a conformable marking sheet, a vehicle window and a film thereof, a vehicle paint protection film, an automotive paint (e.g., a cathodic electrodeposition primer coating and a coating compounded therewith), a solar control film, a solar reflector, a reflective printed label, a UV absorbing glass and a glass coating material, an electrochromic device, a film/a glazing, a windshield, a middle layer, and the like.

In some embodiments, the article of manufacture is an epoxy-reinforced carbon fiber composite material comprising an epoxy-reinforced carbon fiber and an outer layer coating thereof, and the coating comprises a resin coating material and the compound described above. Preferably, the coating material is selected from at least one of an amino acrylic resin coating material, a hydroxyl acrylic resin coating material, a polyester resin coating material, a polyurethane acrylate coating material, a polyurethane resin coating material, an epoxy-modified resin coating material, and a polysiloxane-modified resin coating material.

In some embodiments, the article of manufacture is an optical film or an optical element comprising a coating material resin substrate and the compound described above. Preferably, the resin substrate is selected from at least one of cellulose triacetate (TAC), polycarbonate, polyacrylate, polyethylene naphthalate, polyethylene terephthalate, polyvinyl alcohol, polymethyl methacrylate, cyclopentene, polyimide, polystyrene and derivatives thereof, polyvinyl alcohol, an epoxy resin, polyurethane, and polysilane.

In some embodiments, the article of manufacture is an anti-blue light lens comprising a resin substrate and the compound described above. Preferably, the resin substrate is selected from at least one of aliphatic isocyanate (ADI), polymethyl methacrylate, polycarbonate, polyamide, polymethyl methacrylate, fluorine/silicon-modified polyacrylic acid, and polyisocyanate/polythioether.

In some embodiments, the article of manufacture is an automotive paint comprising a cathodic electrodeposition primer and a coating compounded therewith, and the electrodeposition primer or the coating compounded therewith comprises a resin coating material and the compound described above. Preferably, the cathodic electrodeposition primer coating takes an epoxy resin as a matrix, and the cathodic electrodeposition primer coating further comprises a polyester resin, a polyurethane resin, an epoxy/polyester hybrid resin, an acrylic resin, a polysiloxane resin, an amino acrylic resin, a hydroxyl acrylic resin, a polysiloxane-modified resin, and an epoxy-modified resin.

Yet another aspect of the present invention further provides use of the compound described above, the composition described above, or the polymer material described above as an ultraviolet absorber.

In some embodiments, the compound of the present invention has a significant red shift effect, and is useful in application fields sensitive to the ultraviolet-visible waveband of 280-430 nm, especially application fields with specific shielding requirements for the waveband of 360-430 nm. The compound is used as an ultraviolet absorber and may be used in fields such as epoxy resin-based articles of manufacture and composite articles of manufacture thereof, polyethylene naphthalate-based articles of manufacture, optical adhesives, optical elements, optical films (e.g., optical films taking cellulose triacetate, polycarbonate, polyacrylate, or polyethylene naphthalate as a substrate), optical lenses, anti-blue light articles of manufacture (e.g., anti-blue light lenses, anti-blue light display devices, etc.), reflective sheets and conformable marking sheets, vehicle windows and films thereof, vehicle paint protection films, automotive paints (e.g., cathodic electrodeposition primers and coatings compounded therewith), solar control films, solar reflectors, reflective printed labels, UV absorbing glasses and glass coating materials, electrochromic devices, films/glazings, windshields, middle layers, and the like.

In some embodiments, the present invention is directed to a method of providing an absorption wavelength ranging from 280 nm to 430 nm in a composition, polymer material or article of manufacture as disclosed herein by incorporating a compound as disclosed herein into the composition, polymer material or article of manufacture.

In some embodiments, the present invention is directed to composition, polymer material or article of manufacture as disclosed herein that when tested by a Xenon lamp test per the conditions disclosed herein has an adhesive force of Grade 1 after 14 days or after 21 days.

In some embodiments, the composition, polymer material or article of manufacture incorporating a compound as disclosed herein exhibits a Gardner color scale of about 1 to about 8, about 2 to about 7, about 3 to about 6 or about 3, about 4 or about 5. In certain embodiments, composition, a polymer material or article of manufacture that exhibits a Gardner color scale of about 1 to about 8 without a compound as disclosed herein exhibits a Gardner color scale of about 1 to about 8 with a compound as disclosed herein. In other embodiments, the present invention is directed to methods of maintaining the color of composition, polymer material or article of manufacture with a Gardner color scale of about 1 to about 8 by incorporating a compound as disclosed herein.

The technical solutions provided by the present invention have the following advantages:
(1) Compared with the traditional ultraviolet absorbers, the compound provided by the present invention can overall shift the absorption for the ultraviolet region to the long-wave direction by the novel structural modification, so that the absorption waveband is significantly red-shifted, and the compound has a good light absorption effect in the visible light waveband range of 360-430 nm.
(2) The compound provided by the present invention has excellent solubility in common oligomer resins or active monomers, has better compatibility with solvent-free crosslinking systems used in the preparation of optical devices such as optical adhesive films, optical lenses, and the like, and has better convenience and stronger operability during processing and using.
(3) The compound provided by the present invention also has a lighter color, and can be suitable for light-colored high polymer material articles of manufacture.
(4) The preparation method for the compound provided by the present invention has a simple process and a relatively high yield of the target product.
(5) The compound provided by the present invention is very suitable for being used as an ultraviolet absorber in the field of polymer materials, has excellent protection and shielding effects on visible light within the waveband range of 280-430 nm, has better application performance and wider application range, and is beneficial to the anti-photoaging of the polymer materials, thereby having very important economic and social values.

### DETAILED DESCRIPTION

The present invention provides a compound having a structure represented by formula (I) or formula (II),

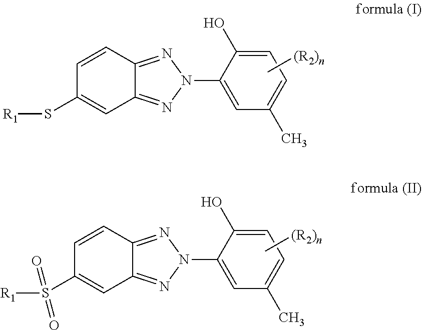

wherein in formula (I) and formula (II), R₁ is selected from C1-C20 linear or branched alkyl, R₂ is selected from C1-C8 linear or branched alkyl, and n is selected from an integer of 1-3.

The present invention further provides a compound having a structure represented by RSUV-1 below (referred to as RSUV-1 in the present invention) or a compound having a structure re resented by RSUV-2 below (referred to as RSUV-2 in the present invention):

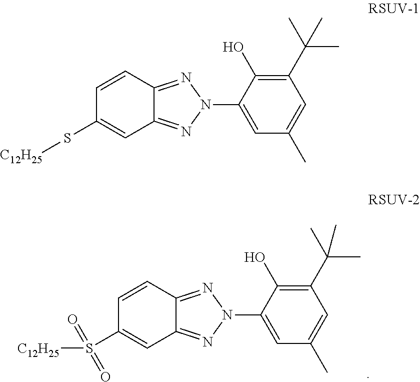

The main structure of benzotriazole is reserved in RSUV-1 or RSUV-2, a thioether bond or sulfonyl is introduced to one side of a benzene ring of benzotriazole, and a substituent on the benzene ring with hydroxy is fixed as methyl to obtain a modified structure; the absorption region of benzotriazole as an ultraviolet absorber is overall shifted to the long-wave direction, and the ultraviolet absorption cut-off wavelength of the modified structure RSUV-2 with sulfonyl is red-shifted from 400-410 nm to the long-wave direction to 430 nm; and the solubility and the compatibility in a special system are also improved.

The compound of the present invention is used for polymer materials sensitive to the ultraviolet-visible waveband of 280-430 nm, such as epoxy resin-based articles of manufacture, epoxy-reinforced carbon fiber coatings, and articles of manufacture based on polyethylene naphthalate (PEN), PEN copolymers, and modified products.

The compound of the present invention is also used in application fields with special shielding requirements for the ultraviolet-visible waveband of 280-430 nm, such as the fields of vehicle windows and films thereof, vehicle paint protection films, optical adhesives, optical elements, optical films, automobile coating materials, and the like.

The present invention further provides an epoxy-reinforced carbon fiber composite material comprising an epoxy-reinforced carbon fiber and an outer layer coating thereof; the coating comprises a resin coating material and the compound described above; preferably, the coating material is selected from at least one of an amino acrylic resin coating material, a hydroxyl acrylic resin coating material, a polyester resin coating material, a polyurethane acrylate coating material, a polyurethane resin coating material, an epoxy-modified resin coating material, and a polysiloxane-modified resin coating material.

Carbon fibers are an emerging material of great interest. At present, carbon fiber reinforced epoxy resin composite materials are the most widely used carbon fiber reinforced materials (CFRMs) having the highest comprehensive indexes such as strength, modulus, etc. However, epoxy resins are themselves susceptible to damage by ultraviolet rays and visible light below 420 nm, causing degradation, and thus affecting their performance. Ultraviolet absorbers with high absorption efficiency in the waveband of 380-420 nm can effectively protect such materials.

Carbon fiber reinforced polymers (CFRPs), also known as carbon fiber reinforced materials (CFRMs), are used in aerospace application fields, for example, due to their high strength-to-weight ratio. Carbon fiber compositions generally contain carbon fibers embedded in thermosetting aromatic epoxy matrixes. These parts are also suitable for use in automotive parts, sporting goods, acoustic assemblies, marine parts, aircraft parts, and the like. The epoxy-reinforced carbon fiber material can be used for interior and exterior decoration parts such as automobile rearview mirrors, empennages, spoilers, and the like.

The carbon fiber reinforced polymer component may be prepared, for example, by well-known molding, vacuum bagging, or compression molding techniques. In the methods, a prepreg may be used. The polymer matrix may, for example, account for about 15-50 wt % of the carbon fiber reinforced polymer component (uncoated).

The present invention further provides an optical film or an optical element comprising a coating material resin substrate and the compound described above; preferably, the resin substrate is selected from at least one of cellulose triacetate (TAC), polycarbonate, polyacrylate, polyethylene naphthalate, polyethylene terephthalate, polyvinyl alcohol, polymethyl methacrylate, cyclopentene, polyimide, polystyrene and derivatives thereof, polyvinyl alcohol, an epoxy resin, polyurethane, and polysilane.

The optical film or the optical device may further comprise catalysts such as crosslinking accelerators, and additives such as antioxidants, ultraviolet absorbers, hindered amine light stabilizers, antistatic agents, dispersion stabilizers, defoaming agents, thickening agents, dispersants, surfactants, and lubricants, as required.

Polyethylene naphthalate (PEN) is a polymer material with excellent comprehensive properties such as chemical stability, mechanical properties, heat resistance, etc., and has relatively good application prospects in the fields of optical elements and the like. However, ultraviolet rays in the waveband of 360-400 nm are prone to causing damage to PEN and composite materials containing PEN, resulting in material degradation. The benzotriazole absorber in the present invention can well and effectively prevent the problem caused by light. In addition to the protection of the substrate of the optical film, it is more important that the photosensitive substance in the optical device also needs to be protected in a wide waveband. For example, the dye in the polarizer needs to be well protected in the ultraviolet waveband of 280-400 nm.

The present invention further provides an anti-blue light lens comprising a resin substrate and the compound described above; preferably, the resin substrate is selected from at least one of aliphatic isocyanate (ADI), polymethyl methacrylate, polycarbonate, polyamide, polymethyl methacrylate, fluorine/silicon-modified polyacrylic acid, and polyisocyanate/polythioether.

The accompanying time of electronic products in modern life is longer and longer, the spectrum emitted by a display light source is very wide, and high-energy blue light in the waveband of 400 nm-445 nm can cause certain damage to the vision health of the user. The benzotriazole derivative in the present invention can absorb the spectrum in the band as much as possible and can reduce the visual injury.

The present invention further provides an automotive paint comprising a cathodic electrodeposition primer and a coating compounded therewith; the cathodic electrodeposition primer layer or the coating compounded (or adhered) therewith comprises a resin coating material and the compound described above; preferably, the cathodic electrodeposition primer coating takes an epoxy resin as a matrix, and the cathodic electrodeposition primer coating further comprise a polyester resin, a polyurethane resin, an epoxy/polyester hybrid resin, an acrylic resin, a polysiloxane resin, an amino acrylic resin, a hydroxyl acrylic resin, a polysiloxane-modified resin, and an epoxy-modified resin.

The coating compounded with the cathodic electrodeposition primer comprises a varnish coating material system, a system of a pigment-containing base color coating material compounded with a varnish, and a pigment-containing single-coating system. The system of a pigment-containing base color coating material compounded with a varnish may be a process system in which the base color coating materials are separately baked; it is also possible to use a process system in which the coating is sprayed by “wet to wet” and finally baked together.

The resin used in the cathodic electrodeposition primer is typically a thermosetting resin used in combination with a crosslinking agent and/or a curing catalyst. Suitable thermosetting resins include an epoxy resin, a polyester resin, a polyurethane resin, an epoxy/polyester hybrid resin, an acrylic resin, a polysiloxane resin, an amino acrylic resin, a hydroxyl acrylic resin, a polysiloxane-modified resin, and an epoxy-modified resin.

The epoxy resin may be cured with dicyandiamide or an acid anhydride. The hydroxyl functional polyester resin can be cured with a polyfunctional isocyanate to form a polyurethane polyester. The acid functional polyester resin can be cured with isocyanurate. The epoxy polyester hybrid can be cured by reacting with each other. The hydroxyl functional acrylic resin can be cured with a polyfunctional isocyanate. The amount of the crosslinking agent or curing agent is dependent on the resin and may, for example, be about 3-20 wt % based on the weight of the resin. The curing is carried out, for example, thermally. The acrylate resin is prepared, for example, from glycidyl acrylate or glycidyl methacrylate.

The present invention includes providing a coating material composition comprising: a coating resin, the compound described above, a hindered amine light stabilizer, and optionally an ultraviolet light absorber, a phenolic antioxidant, and an organic or inorganic pigment.

Most advantageously, the coating material of the present invention is a transparent coating. In addition to the transparent coating material, the coating material of the present invention may also be a pigmented and colored coating. The transparent coating material is defined as being substantially free of pigment.

Other conventional additives may be included in the coating material composition, such as a fluidizing agent, a lubricant, and the like.

The coating material resin is present in the coating material formulation in an amount of about 20-98 wt %, preferably about 30-96 wt %, and more preferably about 50-96 wt %, based on the total amount of the coating material formulation. In other embodiments, the coating material resin is present in the coating material formulation in an amount from any one of about 20%, about 25%, about 30%, about 35%, about 40%, about 45% about 50% or about 55% to any one of about 60%, about 65%, about 70%, about 75%, about 80%, or about 85%, about 90% or about 98%.

The preparation and application of coating materials is well known. The coating material formulation may be applied by spray coating (including electrostatic spraying), dip coating, and roller coating.

The coating material may be applied in one coating to achieve a coating thickness of about 5-200 microns, for example, 20 microns, 40 microns, 50 microns, 60 microns, 80 microns, 100 microns, 120 microns, 150 microns, 180 microns, and any value in between; preferably, the coating thickness is 10-100 microns, more preferably about 20-40 microns. In other embodiments, the coating material may be applied in one coating to achieve a coating thickness from any one of about 5 micron, about 10 micron, about 20 micron, about 30 micron, about 40 micron, about 50 micron, about 60 micron, about 70 micron, about 80 micron, about 90 micron or about 100 micron to any one of about 110 micron, about 120 micron, about 130 micron, about 140 micron, about 150 micron, about 160 micron, about 170 micron, about 180 micron, about 190 micron, or about 200 micron.

The coating material of the present invention exhibit improved durability and excellent exterior weather resistance performance.

In order to make the objects, embodiments, and advantages of the present disclosure more apparent, the present disclosure is further described in detail with reference to the following examples. The specific examples described herein are merely illustrative of the present disclosure and do not constitute any limitation thereon. Moreover, in the following description, descriptions of well-known structures and techniques are omitted so as to avoid unnecessary obscuration of concepts in the present invention. Such structures and techniques are also described in numerous publications.

### Terminology

Unless defined otherwise, all technical and scientific terms used herein have the same meaning as commonly used in the art to which the present disclosure belongs. For the purpose of illustrating the present disclosure, the following definitions will apply, and, where appropriate, terms used in the singular form will also include the plural form and vice versa.

The term “C1-Cn” as used herein includes C1-C2, C1-C3, . . . , and C1-Cn. For example, the “C1-C8” group refers to a moiety having 1-8 carbon atoms, that is, the group contains 1 carbon atom, 2 carbon atoms, 3 carbon atoms, 4 carbon atoms, 5 carbon atoms, 6 carbon atoms, 7 carbon atoms, or 8 carbon atoms. Thus, for example, “C1-C4 alkyl” refers to an alkyl group containing 1-4 carbon atoms, that is, the alkyl is selected from methyl, ethyl, propyl, isopropyl, n-butyl, isobutyl, sec-butyl, and tert-butyl.

The numerical ranges herein, e.g., “1-3”, refer to integers in the given range, that is, 1, 2, or 3.

The term “alkyl” as used herein refers to an optionally substituted linear or optionally substituted branched saturated aliphatic hydrocarbon. The “alkyl” herein may preferably have 1-8 carbon atoms, 1-6 carbon atoms, 1-5 carbon atoms, 1-4 carbon atoms, or 1-3 carbon atoms. Non-limiting examples of the alkyl include methyl, ethyl, n-propyl, isopropyl, 2-methyl-1-propyl, 2-methyl-2-propyl, 2-methyl-1-butyl, 3-methyl-1-butyl, 2-methyl-3-butyl, 2,2-dimethyl-1-propyl, 2-methyl-1-pentyl, 3-methyl-1-pentyl, 4-methyl-1-pentyl, 2-methyl-2-pentyl, 3-methyl-2-pentyl, 4-methyl-2-pentyl, 2,2-dimethyl-1-butyl, 3,3-dimethyl-1-butyl, 2-ethyl-1-butyl, n-butyl, isobutyl, sec-butyl, tert-butyl, n-pentyl, isopentyl, neopentyl, tert-pentyl, hexyl, and longer alkyl groups, such as heptyl, octyl, and the like. In the groups defined herein, for example, when a numerical range appears for “alkyl”, e.g., “C1-C6 alkyl”, it means that an alkyl group may be composed of 1 carbon atom, 2 carbon atoms, 3 carbon atoms, 4 carbon atoms, 5 carbon atoms, or 6 carbon atoms. The alkyl herein also encompasses instances where no numerical range is specified.

Sunlight, in particular ultraviolet radiation from 280 nm to 400 nm, can cause degradation of the plastic, which in turn causes color changes and deterioration of the optical and mechanical properties. Inhibition of photo-oxidative deterioration is important for outdoor applications where long-term durability is mandatory. For example, the absorption of ultraviolet light by polyethylene terephthalate starts at around 360 nm, increases significantly below 320 nm, and is prominent below 300 nm. Polyethylene naphthalate strongly absorbs ultraviolet light in the range of 310-370 nm, the absorption tail extends to about 410 nm, and the absorption maximum value occurs at 352 nm and 337 nm. Chain cleavage occurs in the presence of oxygen and the major photo-oxidation products are carbon monoxide, carbon dioxide, and carboxylic acid. In addition to the direct photolysis of the ester group, the oxidation reaction must also be taken into account, which likewise form carbon dioxide via peroxide free radicals.

The ultraviolet absorbing layer may protect the multilayer optical film by reflecting ultraviolet light, absorbing ultraviolet light, scattering ultraviolet light, or a combination thereof. In general, the ultraviolet absorbing layer can comprise any polymeric composition capable of reflecting, scattering, or absorbing ultraviolet radiation while being subjected to ultraviolet radiation for an extended period of time. Examples of such polymers include PMMA, CoPMMA, organosilicon thermoplastics, fluorine-containing polymers and copolymers thereof and blends thereof Δn exemplary ultraviolet absorbing layer comprises a PMMA/PVDF blend.

Various optional additives may be incorporated into the optical layer to make it ultraviolet absorbing. Examples of such additives include at least one of ultraviolet absorbers, hindered amine light stabilizers, or antioxidants thereof.

Particularly desirable ultraviolet absorbers are red-shifted ultraviolet absorbers (RUVA), which absorb at least 70% (in some embodiments, at least 80%, particularly preferably greater than 90%) of the ultraviolet light in the wavelength region of 180 nm to 400 nm. In other embodiments, the ultraviolet absorbers are red-shifted ultraviolet absorbers (RUVA), which absorb at least 70%, at least 75%, at least 80%, at least 85%, at least 90%, or at least 95% in the wavelength region of 180 nm to 400 nm, 200 nm to 400 nm, 250 nm to 400 nm, 300 nm to 400 nm or 350 nm to 400 nm.

The ultraviolet absorber needs to have the following conditions: The thermal stability is good, the change caused by heat can not occur even in the processing, and the thermal volatility is small; it can strongly absorb ultraviolet (especially with the wavelength of 290-400 nm), has good chemical stability and does not generate adverse reaction with material components in the article of manufacture; the miscibility is good, and it can be uniformly dispersed in materials, does not bloom and does not exude; the absorber has good photochemical stability, does not decompose and does not discolor; it is colorless, nontoxic and odorless; it has immersion cleaning resistance; it is cheap and readily available. Ultraviolet absorbers can be classified into the following groups according to their chemical structures: salicylates, benzophenones, benzotriazoles, substituted acrylonitriles, triazines, and others.

The ultraviolet absorber UV-326 is one of them. The ultraviolet absorber UV-326 is a benzotriazole light stabilizer with the chemical name of 2′-(2′-hydroxy-3′-tert-butyl-5′-methylphenyl)-5-chlorobenzotriazole. UV-326 can absorb the ultraviolet of 280-370 nm, has good stability, small toxicity and no stimulation to humans, is widely applied to coating materials, synthetic rubber, synthetic fibers, photosensitive materials and the like, can effectively improve the light resistance of the materials, and reduces the phenomena of yellowing, aging and the like caused by the excitation of the ultraviolet in sunlight. The ultraviolet absorber UV-326 has the following structure:

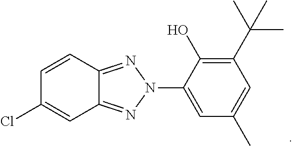 [SMILES: Cc1cc(-n2nc3ccc(Cl)cc3n2)c(O)c(C(C)(C)C)c1]

Commercially available red-shifted ultraviolet absorber Carboprotect also belongs to the benzotriazole (BTZ) ultraviolet absorbers, is solid, has a melting point of 132-136° C. and a molecular weight (g/mol) of 560, is a red-shifted ultraviolet absorber, and protects the aromatic hydrocarbon epoxy resin system; it is recommended for carbon or glass fiber reinforced composite materials; it can be red-shifted to 420 nm wavelength. Carboprotect has the following:

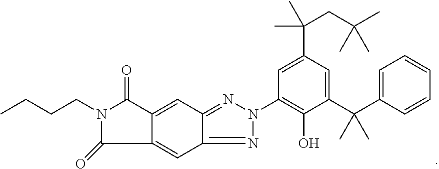 [SMILES: CCCCN1C(=O)c2cc3nn(-c4cc(C(C)(C)CC(C)(C)C)cc(C(C)(C)c5ccccc5)c4O)nc3cc2C1=O]

### BRIEF DESCRIPTION OF THE DRAWINGS

FIG. 1 shows the synthetic routes of the target products RSUV-1 and RSUV-2.

FIG. 2 shows an LC-MS spectrum of the target product RSUV-1.

FIG. 3 shows the hydrogen nuclear magnetic resonance spectroscopy of the target product RSUV-1.

FIG. 4 shows an LC-MS spectrum of the target product RSUV-2.

FIG. 5 shows the hydrogen nuclear magnetic resonance spectroscopy of the target product RSUV-2.

### LIST OF ITEMS

1. A compound having a structure represented by formula (I) or formula (II),

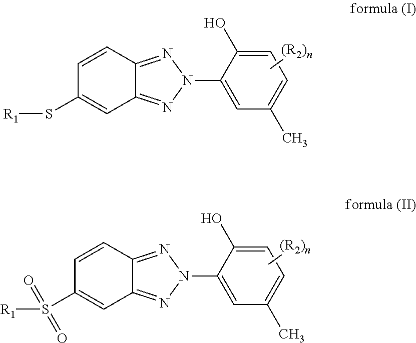

wherein in formula (I) and formula (II), R₁ is selected from C1-C20 linear or branched alkyl, R₂ is selected from C1-C8 linear or branched alkyl, and n is selected from an integer of 1-3.

2. The compound according to item 1, wherein the compound has a structure represented by formula (III) or formula (IV),

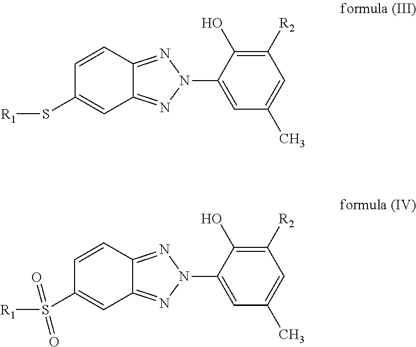

wherein in formula (III) and formula (IV), R₁ is selected from C1-C20 linear or branched alkyl, and R₂ is selected from C1-C8 linear or branched alkyl.

3. The compound according to item 1 or 2, wherein in formula (I), formula (II), formula (III), and/or formula (IV), R₁ is selected from C5-C20 linear or branched alkyl, preferably C7-C15 linear or branched alkyl, and R₂ is selected from C2-C6 linear or branched alkyl; preferably, the compound has a structure represented by RSUV-1 or RSUV-2:

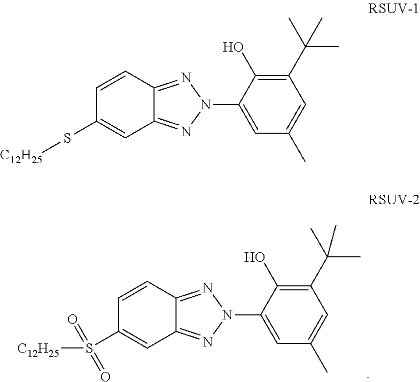

4. The compound according to item 1, wherein the compound has an absorption wavelength ranging from 280 nm to 430 nm.

5. A composition comprising the compound according to any one of items 1-4.

6. The composition according to item 5, wherein the composition further comprises one or more additives; preferably, the additive comprises one or more of a hindered amine light stabilizer, an additional ultraviolet absorber, an antioxidant, an emulsifier, a nucleating agent, a toughening agent, a lubricant, an anti-blocking agent, a filler, a dye, a pigment, a fluorescent brightener, a flame retardant, an antistatic agent, or a foaming agent.

7. A polymer material, wherein the polymer material comprises the compound according to any one of items 1-4 or the composition according to item 5 or 6, and an organic material susceptible to degradation induced by oxygen, heat, or light; preferably, the organic material is at least one resin of polyester, polyurethane, polyacrylic acid, polycarbonate, epoxy and modified resins thereof, phenolic resin, polyamide, polyimide, polystyrene and derivatives thereof, polysilane, polysiloxane and modifications thereof, poly(vinyl butyral), amino acrylic acid, hydroxyl acrylic acid, polyurethane acrylate, polycyanoacrylate, polyacrylate, ethylene/acrylic acid copolymers and salts thereof (ionomers), poly(vinyl alcohol), cellulose triacetate, polycarbonate, polyethylene naphthalate, polyethylene terephthalate, polyvinyl alcohol, polymethyl methacrylate, polycyclopentene, aliphatic isocyanate, polymethyl methacrylate, fluorine/silicon-modified polyacrylic acid, and polyisocyanate/polythioether.

8. An article of manufacture comprising the compound according to any one of items 1-4, the composition according to item 5 or 6, or the polymer material according to claim 7.

9. The article of manufacture according to item 8, wherein the article of manufacture is selected from an epoxy resin-based article of manufacture and composite articles of manufacture thereof, a polyethylene naphthalate-based article of manufacture, an optical adhesive, an optical element, an optical film, an optical lens, an anti-blue light article of manufacture, a reflective sheet and a conformable marking sheet, a vehicle window and a film thereof, a vehicle paint protection film, an automotive paint, a solar control film, a solar reflector, a reflective printed label, a UV absorbing glass and a glass coating material, an electrochromic device, a film/a glazing, a windshield, or a middle layer; preferably, the article of manufacture is an epoxy-reinforced carbon fiber composite material comprising an epoxy-reinforced carbon fiber and an outer layer coating thereof, the coating comprises a resin coating material and the compound according to any one of items 1-4; the coating material is preferably selected from at least one of an amino acrylic resin coating material, a hydroxyl acrylic resin coating material, a polyester resin coating material, a polyurethane acrylate coating material, a polyurethane resin coating material, an epoxy-modified resin coating material, and a polysiloxane-modified resin coating material; preferably, the article of manufacture is an optical film or an optical element comprising a coating material resin substrate and the compound according to any one of items 1-4; the resin substrate is preferably selected from at least one of cellulose triacetate, polycarbonate, polyacrylate, polyethylene naphthalate, polyethylene terephthalate, polyvinyl alcohol, polymethyl methacrylate, polycyclopentene, polyimide, polystyrene and derivatives thereof, an epoxy resin, polyurethane, and polysilane; preferably, the article of manufacture is an anti-blue light lens comprising a resin substrate and the compound according to any one of items 1-4; the resin substrate is preferably selected from at least one of aliphatic isocyanate, polymethyl methacrylate, polycarbonate, polyamide, polymethyl methacrylate, fluorine/silicon-modified polyacrylic acid, and polyisocyanate/polythioether; preferably, the article of manufacture is an automotive paint comprising a cathodic electrodeposition primer and a coating compounded therewith; the electrodeposition primer or the coating compounded therewith comprises a resin coating material and the compound according to any one of items 1-5; the cathodic electrodeposition primer coating takes an epoxy resin as a matrix, and the cathodic electrodeposition primer coating further comprises a polyester resin, a polyurethane resin, an epoxy/polyester hybrid resin, an acrylic resin, a polysiloxane resin, an amino acrylic resin, a hydroxyl acrylic resin, a polysiloxane-modified resin, and an epoxy-modified resin.

10. Use of the compound according to any one of items 1-4, the composition according to claim 5 or 6, or the polymer material according to claim 7 as an ultraviolet absorber.

### Example

The following examples take the three groups of existing products as comparative products, including:
(1) comparative product 1: a conventional ultraviolet absorber (hereinafter referred to as UV-326);
(2) comparative product 2: Commercially available, red-shifted ultraviolet absorber Carboprotect from BASF
(3) comparative product 3: the structure is as follows (hereinafter referred to as

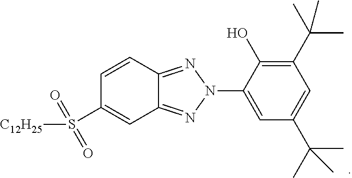

Reagents without specified manufacturers used in the examples are conventional products that are commercially available.

### Example 1: Synthesis Method

Two target products RSUV-1 and RSUV-2 were obtained according to the synthetic route shown in FIG. 1. The details are as follows:

### First-Step of the Reaction (Step 1):

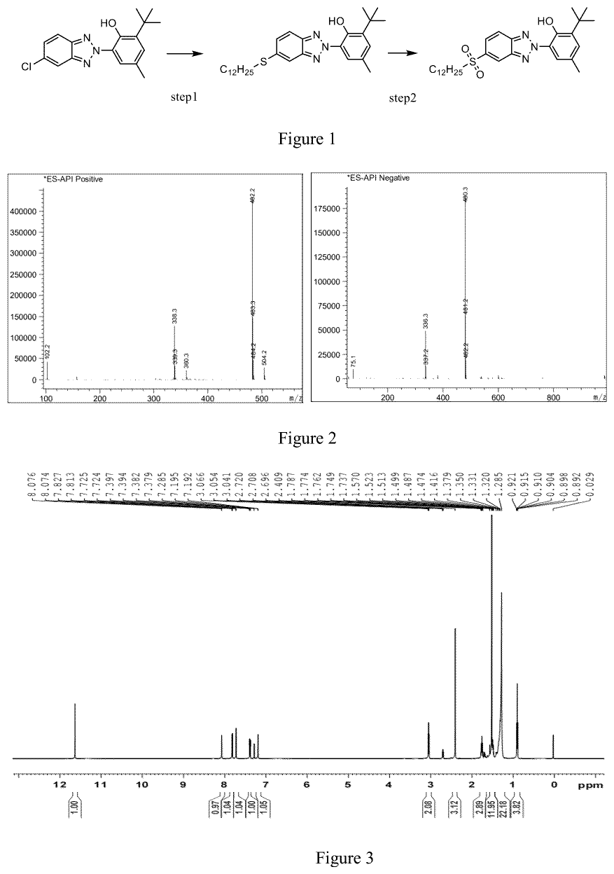

UV326 (200.0 g, 0.63 mol), dodecyl mercaptan (NDM, 154.5 g, 0.76 mol), potassium carbonate (121.5 g, 0.89 mol) and N,N-dimethylformamide (DMF, 1000 mL) were sequentially added to a 2000 mL four-neck round-bottom flask, and the mixture was warmed to reflux. After 5-7 h of reaction, the starting material had been completely consumed, and the reaction was terminated; the mixture was cooled to 110-120° C. and filtered under vacuum to remove the inorganic salt in the reaction solution. The mother solution was distilled under reduced pressure, and DMF was evaporated; 1000 mL of dimethylbenzene was then added to dissolve the material, a small amount of acetic acid was added at 80-90° C., the pH of the solution was adjusted to be neutral, and the solution was washed with washing water for 3-4 times with each time 200 mL; after the washing was completed, the mixture was warmed, refluxed and dehydrated until no water was evaporated. If the sulfonylation product RSUV-2 was produced, the reaction product in this step can be directly subjected to the next reaction without purification; if the thioether product RSUV-1 was required, dimethylbenzene was removed by reduced pressure distillation, the mixture was then cooled to 50° C., 1000 mL of methanol/dimethylbenzene mixed solution was added for crystallization, and the mixture was cooled to room temperature, then crystallized for 3 h with the temperature maintained at this temperature, filtered under vacuum and dried to give a yellow solid powder (303.5 g, yield 97%). LC-MS detection results: [M+1]=482.2, [M−1]=480.2, and the spectrum is shown in FIG. 2. The hydrogen nuclear magnetic resonance spectroscopy is shown in FIG. 3.

### Second-Step of the Reaction (Step 2)

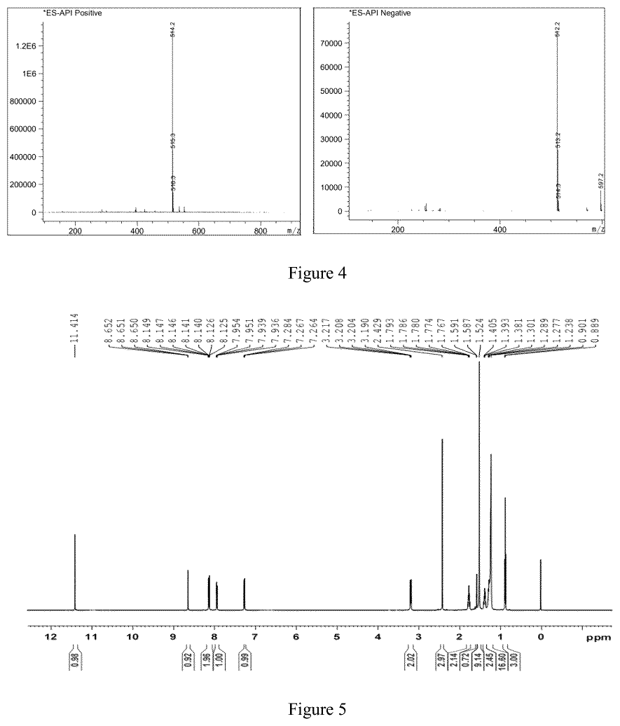

The solution refluxed and dehydrated described above was cooled to room temperature. Formic acid (66.4 g, 1.27 mol) and sodium tungstate (catalytic amount) were added, and hydrogen peroxide (194 g, 2.86 mol) was added dropwise. After the dropwise addition was completed, the mixture was warmed to 50-70° C. and reacted for 3-5 h with the temperature maintained at this temperature, and the reaction was completed. The mixture was left to stand. The lower aqueous phase was separated out, a small amount of an aqueous sodium sulfite solution was then added to remove the remaining hydrogen peroxide, and the mixture was washed with saturated brine and pure water 3 times, respectively. The mixture was warmed to 110-140° C., distilled under reduced pressure to remove dimethylbenzene, and cooled to 50° C. 1000 mL of methanol was added for crystallization, and the mixture was cooled to room temperature, then crystallized for 3 h with the temperature maintained at this temperature, filtered under vacuum and dried to give a yellow solid powder (264 g, yield 76%). LC-MS detection results: [M+1]=514.2, [M−1]=512.2, and the spectrum is shown in FIG. 4. The hydrogen nuclear magnetic resonance spectroscopy is shown in FIG. 5.

### Example 2 Absorption Spectrum

Solutions in chloroform (10 mg/L) of UV absorbers 1 (UV-326), RSUV-1, RSUV-2, and Carboprotect were prepared. The absorption spectra were measured using an ultraviolet-visible spectrophotometer, and the instrument model is (UV-2600, Shimadzu corporation, Japan). The absorption spectra were compared in Table 1 below.

TABLE 1

| | | | | | | Characteristic value | |
|---|---|---|---|---|---|---|---|
| | | | | | | of absorption spectrum | |
| | | | | | | Maximum | |
| | Peak area ratio in waveband | | | | | absorption peak | Absorption |
| | 280-360 | 361-380 | 381-400 | 401-450 | 280-450 | in red shift | cut-off |
| | nm | nm | nm | nm | nm | direction/nm | wavelength/nm |
| UV-326 | 75.1% | 18.0% | 6.5% | 0.4% | 100.0% | 356 | 410 |
| RSUV-1 | 58.1% | 26.5% | 13.6% | 1.8% | 100.0% | 369 | 416 |
| RSUV-2 | 65.4% | 18.1% | 11.8% | 4.7% | 100.0% | 370 | 430 |
| Carboprotect | 56.2% | 14.6% | 18.5% | 10.7% | 100% | 374 | 435 |

As can be seen from Table 1, the ultraviolet absorbers of the products RSUV-1 and RSUV-2 prepared according to the present invention were red-shifted overall to the long-wave direction. RSUV-1 has significantly stronger absorption in the waveband of 360-450 nm than that of the conventional product (comparative product 1). The cut-off wavelength of RSUV-2 was at 430 nm, which can provide more anti-photoaging protection for substrates sensitive to the 360-430 nm waveband. In addition Carboprotect UV absorber's higher absorption in the longer wavelength, compared to RSUV-1 and RSUV-2 contributes to higher initial color of this UVA which is unsuitable in several critical color sensitive applications where this additive can impart higher initial color. Thus RSUV-1 and RSUV-2 are sufficiently red shifted to give the desired protection from longer wavelength UV light without the detrimental higher initial color in several end uses.

### Example 3 Solubility/Compatibility Test

For many optical devices, such as optical adhesive films and optical lenses, solvent-free crosslinking systems are used in the formation of the optical devices. The system can avoid the problems of precision reduction caused by shrinkage, device pollution caused by solvent and the like. However, in the case of an auxiliary agent such as an ultraviolet absorber, it is only dissolved by an oligomer resin or an active monomer. Therefore, there are high demands on the solubility or compatibility of the ultraviolet absorber in these substances.

The comparative product 3 (EX16) and RSUV-1 and RSUV-2 prepared according to the present invention were tested for solubility in typical oligomer resins (cyclohexanedimethylene diisocyanate (H6XDI, purity≥99.7%, Wanhua Chemical), hexamethylene diisocyanate (HDI) trimer (Desmodur N3300, Covestro), and tripropylene glycol diacrylate (≥80% (GC), Aladdin Reagent).

Test method: The mass of soluble ultraviolet absorber in 100 g of oligomer when the ambient temperature is 20° C. The test results are shown in Table 2 below.

TABLE 2
Results of solubility test

| | EX16/g | RSUV-1/g | RSUV-2/g |
|---|---|---|---|
| Cyclohexanedimethylene | 1.2 | 4.2 | 3 |
| diisocyanate (H₆XDI) | | | |
| Hexamethylene diisocyanate | 1.6 | 4.6 | 3.5 |
| (HDI) trimer | | | |
| Tripropylene glycol diacrylate | 2.6 | 3.9 | 3.8 |

As can be seen from Table 2, RSUV-1 and RSUV-2 prepared according to the present invention have better solubility as ultraviolet absorbers in several types of typical oligomer resins than that of the comparative compound EX16, and thus have better compatibility with a solvent-free crosslinking system, better convenience in addition and use, and stronger workability.

### Example 4 Color Test

2 g of the comparative product 2 (Carboprotect) and RSUV-1 and RSUV-2 prepared according to the present invention were separately dissolved in 18 g of dimethylformamide (DMF) solvent. The completely dissolved solution was transferred to a cuvette, and the Gardner color scale value transmitted by the solvent was measured. The instrument was a Ci7600 bench spectrophotometer (X-rite, the United States). The test results are shown in Table 3 below.

TABLE 3

| | Gardner color scale |
|---|---|
| Carboprotect | 11.5 |
| RSUV-1 | 3.0 |
| RSUV-2 | 5.2 |

As can be seen from Table 3, the commercially available, red-shifted ultraviolet absorber Carboprotect is very dark in color and has a major effect on the article of manufacture finally used, particularly a light-colored article of manufacture. The color of RSUV-1 and RSUV-2 prepared according to the present invention as ultraviolet absorbers is obviously lighter, especially the color of RSUV-1, so that they have relatively great advantages for application.

From Examples 2-4, it can be found that compared with the conventional ultraviolet absorber UV-326, the commercially available, red-shifted ultraviolet absorber Carboprotect, and EX16 with a similar structure, the ultraviolet absorber provided by the present invention has significantly more excellent overall performance, especially in the aspects of light absorption performance, solubility performance, chroma, etc., and thus has a wider application prospect.

### Example 5 Use of Ultraviolet Absorber in Epoxy-Reinforced Carbon Fiber Coating

The example relates to a coating material for coating the surface of epoxy-reinforced carbon fibers, and the coating material is based on an isocyanate-curing two-component acrylic varnish system: In the component A (shown in Table 4) in the two-component acrylic varnish, different ultraviolet absorbers were separately added; Desmodur N3300 (Covestro) was used for the component B. When in use, according to the weight percentage, component A:component B=100:21, and the two components were uniformly mixed and sprayed. In this ratio, according to the molar weight, isocyanate

TABLE 4
Formulation of component A

| | | | Experi- | Experi- |
|---|---|---|---|---|
| | | Control | mental | mental |
| | Blank | group 1 | group 1 | group 2 |
| Nuplex hydroxyl acrylic resin | 60 | 60 | 60 | 60 |
| Setalux 1274 BA-70/wt % | | | | |
| Leveling agent BYK 378/wt % | 0.2 | 0.2 | 0.2 | 0.2 |
| Methyl amyl ketone/wt % | 12 | 12 | 12 | 12 |
| Butyl acetate/wt % | 14.15 | 14.15 | 14.15 | 14.15 |
| Toluene/wt % | 12 | 12 | 12 | 12 |
| UV-326/wt % | 0 | 1 | 0 | 0 |
| RSUV-1/wt % | 0 | 0 | 1 | 0 |
| RSUV-2/wt % | 0 | 0 | 0 | 1 |
| Light stabilizer Riasorb UV- | 0.6 | 0.6 | 0.6 | 0.6 |
| 123/wt % | | | | |
| DBTDL (5% solution)/wt % | 0.05 | 0.05 | 0.05 | 0.05 |

Note:
DBTDL (dibutyltin dilaurate) was used as a catalyst and was prepared as a 5% methyl amyl ketone solution.

### Test Method:

(1) The coating materials in Table 4 were mixed according to the specified ratio and sprayed on an epoxy-reinforced carbon fiber plate (purchased from Weisheng New Material Technology Co., Ltd.) with the film thickness of 30-35 microns and baked in an oven at 80° C. for 45 min until curing and film formation; the test plate was placed in dark at room temperature for one week to be tested.
(2) The test plate was placed in a xenon lamp aging test chamber (model: Atlas Ci4400). For test standard, reference was made to SAE J2527. The test plate was taken out every 2000 h to measure the 20 degree gloss of the coating. The colorimeter was: BYK three-angle glossmeter (model: micro-TRI-gloss). After 6000 h of testing, the adhesive force was tested, and the reference standard was ISO 2409-2013. The test results are shown in Table 5 below.

TABLE 5

| | 20° gloss after aging | | | | |
|---|---|---|---|---|---|
| Ultraviolet | | 2000 | 4000 | 6000 | Adhesive force after 6000 |
| absorber | Initial | h | h | h | h of Xenon Exposure |
| Blank | 91.7 | 68.9 | 50.4 | 24.5 | Rate 5, all dropped |
| UV-326 | 91.2 | 71.2 | 64.4 | 42.6 | Rate 5, all dropped |
| RSUV-1 | 90.4 | 78.5 | 65.3 | 50.4 | Rate 4, a large area dropped |
| RSUV-2 | 91.0 | 85.0 | 68.2 | 55.9 | Rate 2, a small amount |
| | | | | | dropped |

As can be seen from Table 5, RSUV-1 and RSUV-2 prepared according to the present invention are effective as ultraviolet absorbers for improving the gloss and adhesive force of epoxy-reinforced carbon fiber coatings compared with the conventional ultraviolet absorber UV-326. The protective effect of RSUV-1 is improved to a certain extent compared with that of the UV-326, and the protective effect of RSUV-2 is significantly improved. Meanwhile, the color of RSUV-2 is lighter, the influence thereof on a coating or other polymer articles of manufacture is smaller, and the application field is wider.

### Example 6 Comparison of UV 326, RSUV2 and Carboprotect Epoxy-Reinforced Carbon Fiber Coating

TABLE 6
Formulation of component A

| | | | Experi- | Experi- |
|---|---|---|---|---|
| | | Control | mental | mental |
| | Blank | group 1 | group 1 | group 2 |
| Nuplex hydroxyl acrylic resin | 60 | 60 | 60 | 60 |
| Setalux 1274 BA-70/wt % | | | | |
| Leveling agent BYK 378/wt % | 0.2 | 0.2 | 0.2 | 0.2 |
| Methyl amyl ketone/wt % | 12 | 12 | 12 | 12 |
| Butyl acetate/wt % | 14.15 | 14.15 | 14.15 | 14.15 |
| Toluene/wt % | 12 | 12 | 12 | 12 |
| UV-326/wt % | 0 | 1.4 | 0 | 0 |
| RSUV-2/wt % | 0 | 0 | 1.4 | 0 |
| Carboprotect wt % | 0 | 0 | 0 | 1.4 |
| Light stabilizer Riasorb UV- | 0.6 | 0.6 | 0.6 | 0.6 |
| 292/wt % | | | | |
| DBTDL (5% solution)/wt % | 0.05 | 0.05 | 0.05 | 0.05 |

Note:
DBTDL (dibutyltin dilaurate) was used as a catalyst and was prepared as a 5% methyl amyl ketone solution

The coating materials in Table 6 were mixed according to the specified ratio and sprayed on an epoxy-reinforced carbon fiber plate (purchased from Weisheng New Material Technology Co., Ltd.) with the film thickness of 45 microns and baked in an oven at 80° C. for 45 min until curing and film formation; the test plate was placed in dark at room temperature for one week to be tested.

(2) The test plate was placed in a xenon lamp aging test chamber (model: Atlas Ci4400), and exposures were done using ISO 11341(2004) protocol. The test plate was taken out periodically to measure adhesion of the coating, as per ASTM D 3359-09, The test results are shown in Table 7 below.

TABLE 7
Adhesion Rating after hours of Xenon Exposure

| UV Absorber | 1000 hours | 2400 hours | 3200 hours |
|---|---|---|---|
| Unstabilzed | 5B | — | — |
| UV-326 | 1B | 2B | 5B |
| RSUV-2 | 0B | 2B | 2B |
| Carboprotect | 0B | 5B | |

Adhesion test:
0B: No delamination
1B: <5% delamination
2B: 5%~15% delamination
3B: 15%~35% delamination
4B: 35~65% delamination
5B: >65% delamination

The results above shows the superior performance of the UV absorber of the current invention when compared to UV 326 and the current state of the art carboprotect UV absorbers. Both UV 326 and carboprotect fail with extensive delamination after 2400 hours with a 5B rating compared to the RSUV-2, which shows a delamination rating of 2B after 3200 hours.

The UV absorber RSUV-2 of the current invention is able protect the coating on light unstable CFRM substrate and prevent delamination better than the commercial control.

### Example 7 Use of Ultraviolet Absorber in Optical Film

The optical film is a relatively wide range of applications, and as a main carrier substrate of the optical films, polymer films are required to have excellent optical properties. Such polymers include cellulose triacetate (TAC), polycarbonate, polyacrylate, polyethylene naphthalate (PEN), and the like. In order to achieve certain optical properties, such as full blocking of the ultraviolet light region (280-400 nm), or to protect certain specific devices therein from damage caused by ultraviolet light with a specific waveband (as mentioned above, the PEN film is in the sensitive waveband of 360-400 nm), ultraviolet absorbers are usually added. The example investigates the ultraviolet blocking properties of the ultraviolet absorbers.

(1) The Materials were Prepared According to the Following Formulation:
Cellulose triacetate (acetyl degree 60-61%): 100 parts
Dichloromethane: 400 parts
Methanol: 40 parts
Biphenyl diphenyl phosphate: 6 parts
Ultraviolet absorber UV-326/RSUV-1/RSUV-2: 0.5 parts
   (2) All the materials were added according to the formulation ratio described above, and the mixture was sealed and stirred for 3 h until a uniform and transparent adhesive solution was formed. The adhesive solution was coated into a film and then dried in an oven at 125° C. for 30 min. A 200 micron TAC film was prepared.

### (3) Testing of UV Absorption Value of Optical Adhesive Film

The optical adhesive film prepared above was cut into a test piece of 25 mm×60 mm. The absorption spectrum was measured using an ultraviolet-visible spectrophotometer, and the instrument model is (UV-2600, Shimadzu corporation, Japan). The absorption characteristic values of the optical films are shown in Table 6 below.

TABLE 6

| | | | |
|---|---|---|---|
| Technical requirements | UV-326 | RSUV-1 | RSUV-2 |

| | | | | |
|---|---|---|---|---|
| 300 nm | ≤0.2% | 0.02 | 0.02 | 0.02 |
| 380 nm | ≤0.2% | 0.12 | 0.08 | 0.16 |
| 400 nm | ≤5% | 18.5 | 4.4 | 0.42 |
| 550 nm | ≥90% | 92.4 | 92.4 | 91.9 |

As can be seen from Table 6, RSUV-1 and RSUV-2 prepared according to the present invention can better cover the desired waveband range of the optical adhesive film as ultraviolet absorbers, simultaneously have no negative absorption to higher waveband ranges, and do not affect the normal optical requirements.

### Example 8 Use of Ultraviolet Absorber in Anti-Blue Light Lens

The wavelength range of blue light is 380 nm-500 nm in the visible wavelength range. Studies have shown that short-wave blue light with the wavelength between 400-455 nm has damage to retinal pigment epithelial cells due to short wavelength and high energy, and can cause vision injury such as macular degeneration. However, mid- and long-wave blue light between 445-500 nm belongs to the beneficial blue light because it participates in a mechanism of biorhythm called “circadian rhythm cycle”. If the high-energy blue light between 400-445 nm described above can be shielded in the display or the glasses, the user of the electronic product can be directly benefited in health. Taking an anti-blue light lens as an example, blue light can be effectively filtered off generally by means of film plating and addition of a blue-violet light absorber. Although most of the blue light can be effectively filtered off by the method of film reflection, the filtering effect on the blue and violet light with low wavelength (380 nm-420 nm) is relatively limited. Then the red-shifted ultraviolet absorber with better absorption effect at 380-420 nm can play a better anti-blue light effect.

The lens has a wide variety of resins, including polycarbonate, polymethacrylate, and polyurethane. Among them, polyurethane optical resins are excellent in overall performance and are an important development direction of new optical resins in recent years. The example demonstrated the effect of the red-shifted ultraviolet absorber of the present invention in a polyurethane resin.

### (1) Starting Material Ratio

Cyclohexanedimethylene diisocyanate (H6XDI, purity 99.7%, Wanhua Chemical): 500 g
2,3-dithio(2-mercapto)-1-propanethiol (purity 98%, Sigma-Aldrich): 223.5 g
Pentaerythritol tetrakis(3-mercaptopropionate) (purity 98%, Sigma-Aldrich): 314.5 g
Dibutyltin dichloride (purity 99%, Xiya Reagent) catalyst: 1.5 g
Ultraviolet absorber UV-329 (Rianlon Corporation): 1.5 g
Test sample: UV-326/RSUV-2: 1.5 g

### (2) Lens Manufacturing Process

Dibutyltin dichloride, the ultraviolet absorber UV-329, a test sample, and cyclohexanedimethylene diisocyanate (H6XDI) were added according to the formulation ratio described above under stirring, mixed and dissolved.

After the dissolution was completed, adding 2,3-dithio(2-mercapto)-1-propanethiol and pentaerythritol tetrakis(3-mercaptopropionate) were added. All the materials were stirred, mixed, filtered by a filter membrane, injected into a special lens mold, and degassed under reduced pressure for 1 h. The molar ratio of NCO to SH groups in the resin was 1:1.

The degassed liquid resin was placed in an oven, slowly warmed to 120° C. for polymerization and curing, and maintained at 120° C. for 2 h to fully cure the resin; after the first curing, the resin was naturally cooled to room temperature and subjected to a secondary curing at room temperature. After 48 h, demolding was performed to give the resin lens.

### (3) Blue Light Blocking Rate Test

The transmittance of each waveband of the prepared lens described above was tested by referring to GB/T 38120-2019 Technical requirements on application of light health and light safety of coating for protection against blue light. The results are shown in Table 7 below.

TABLE 7

| | | Transmittance in ultraviolet region | | Transmittance in blue light region | | |
|---|---|---|---|---|---|---|
| Lens number | Test sample | 280-380 nm | 380-400 nm | 400-420 nm | 420-445 nm | 445-500 nm |
| 1 | UV-326 | 0% | 4.20% | 83.50% | 94.34% | 95.20% |
| 2 | RSUV-2 | 0% | <0.02% | 8.61% | 68.70% | 94.60% |

As can be seen from Table 7, after RSUV-2 prepared according to the present invention is added as an ultraviolet absorber, the absorption of the lens in the blue light region is significantly enhanced, and high-energy harmful blue light can be effectively blocked.

### Example 9 Use of Ultraviolet Absorber in Cathodic Electrodeposition Paints and Compounded Coatings Thereof

Cathodic electrodeposition paints (Electro-coat) is widely used in the fields of industrial equipment, automobiles and the like, and provides excellent corrosion resistance and chemical resistance for metal substrates in the fields. The main resin of the cathodic electrodeposition paint is generally selected from resins including an epoxy resin, a polyester resin, a polyurethane resin, an epoxy/polyester hybrid resin. Among them, the epoxy ester or epoxy-modified resin is a core component. Epoxy resins are very sensitive to ultraviolet light. Not only the conventional ultraviolet range, but even near ultraviolet waveband can cause damage to epoxy resins. The absorption waveband of RSUV-2 provided by the present invention exceeds the covering wavelength of the conventional ultraviolet absorber, and particularly can provide a relatively good ultraviolet shielding effect at 380-430 nm, so that a better protection effect can be provided for the cathodic electrodeposition paint.

### (1) Starting Material Ratio

A typical one-component solvent varnish (composition formulation shown in Table 8 below) was selected:

TABLE 8
Composition of acrylic amino varnish for automobile

| Material | Blank | Control group 1 | Experimental group 1 |
|---|---|---|---|
| Setalux 1766 | 32 | 32 | 32 |
| Setalux 1795 | 10 | 10 | 10 |
| Setalux 91795 | 6.6 | 6.6 | 6.6 |
| Setal 168 | 2.5 | 2.5 | 2.5 |
| Cymel 303 | 2 | 2 | 2 |
| Cymel 1168 | 7 | 7 | 7 |
| BYK 378 | 0.5 | 0.5 | 0.5 |
| BYK 306 | 0.3 | 0.3 | 0.3 |
| UV-326 | / | 1.4 | / |
| RSUV-2 | / | / | 1.4 |
| UV-123 | / | 0.6 | 0.6 |
| Methyl amyl ketone | 6 | 6 | 6 |
| Butyl acetate | 15.5 | 14.5 | 14.5 |
| 100# solvent oil | 17.4 | 16.4 | 16.4 |
| Nacure 5225 | 0.2 | 0.2 | 0.2 |

Note:
Setalux 1766, Setalux 1795, Setalux 91795, and Setal 168: hydroxyl acrylic acid resins and anti-sagging resins, purchased from Zhanxin Resin (China) Co., Ltd.; Cymel 303 and Cymel 1168: amino resins, purchased from Changxin Resin (Guangdong) Co., Ltd.; BYK 378 and BYK 306: organosilicon leveling agents, purchased from BYK Auxiliary Agent (Shanghai) Co., Ltd.; Nacure 5225: acid catalyst, purchased from King Industries Inc., the United States

### (2) Test Method

Based on the acrylic amino varnish formulation described above, different types of light stabilizers were added to give the following three groups of test coating material samples. The coating material test sample plate was made by spraying according to the following method: The substrate was a steel plate with an electrodeposition coating (purchased from ACT Test Panels LLC., the United States, ECOAT: U32AD800). The three groups of varnishes described above were separately sprayed, the film thickness was 40 m, the three groups were leveled at room temperature for 10 min, and the plates were baked in an oven at 140° C. for 30 min.

Xenon lamp test conditions were as follows: The test plate was placed in a xenon lamp aging test chamber (model: Atlas Ci4400 xenon lamp aging test chamber). For test standard, reference was made to ISO 11341 (2004). The test plate was taken out every 7 days to measure the adhesive force of the coating, and the reference standard was ISO 2409-2013. The test results are shown in Table 9 below.

TABLE 9
Test results of adhesive force after xenon lamp aging

| | Day 7 | Day 14 | Day 21 | Day 28 |
|---|---|---|---|---|
| Blank | Grade 5, mostly | / | / | / |
| | dropped | | | |
| Control | Grade 1 | Grade 3 | Grade 5, mostly | / |
| group 1 | | | dropped | |
| Experimental | Grade 1 | Grade 1 | Grade 1 | Grade 3 |
| group 1 | | | | |

As can be seen from the results in Table 9, RSUV-2 prepared according to the present invention can maintain the adhesive force between the varnish and the cathodic electrodeposition paint for a long period of time.

As can be seen from Examples 5-8, RSUV-1 and RSUV-2 provided by the present invention can be well utilized in various fields as ultraviolet absorbers, and have wide application range and strong industrial practicability.

What is claimed is:

1. A compound having a structure represented by formula (I) or formula (II), 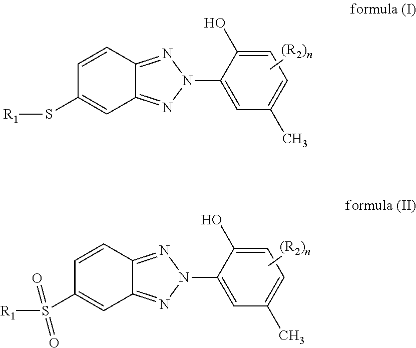
wherein in formula (I) and formula (II), R₁ is selected from C1-C20 linear or branched alkyl, R₂ is selected from C1-C8 linear or branched alkyl, and n is selected from an integer of 1-3.

2. The compound according to claim 1, wherein the compound has a structure represented by formula (III) or formula (IV), 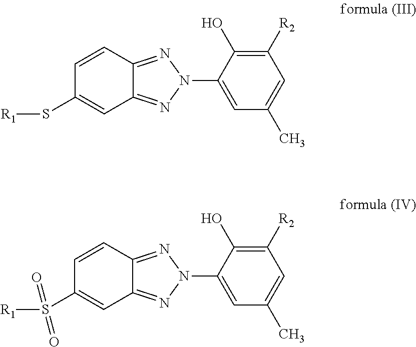
wherein in formula (III) and formula (IV), R₁ is selected from C1-C20 linear or branched alkyl, and R₂ is selected from C1-C8 linear or branched alkyl.

3. The compound according to claim 1, wherein in formula (I), formula (II), formula (III), and/or formula (IV), R₁ is selected from C5-C20 linear or branched alkyl, preferably C7-C15 linear or branched alkyl, and R₂ is selected from C2-C6 linear or branched alkyl.

4. The compound according to claim 1 having a structure represented by RSUV-1 or RSUV-2:

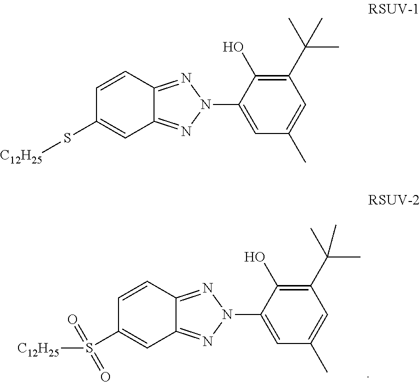

5. The compound according to claim 1 having a structure represented by RSUV-2:

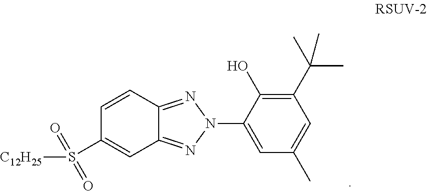

6. A composition comprising the compound according to claim 1.

7. The composition according to claim 6, wherein the composition further comprises one or more additives selected from one or more of a hindered amine light stabilizer, an additional ultraviolet absorber, an antioxidant, an emulsifier, a nucleating agent, a toughening agent, a lubricant, an anti-blocking agent, a filler, a dye, a pigment, a fluorescent brightener, a flame retardant, an antistatic agent, or a foaming agent.

8. A polymer material, wherein the polymer material comprises the compound according to claim 1, and an organic material selected from at least one resin of polyester, polyurethane, polyacrylic acid, polycarbonate, epoxy and modified resins thereof, phenolic resin, polyamide, polyimide, polystyrene and derivatives thereof, polysilane, polysiloxane and modifications thereof, poly(vinyl butyral), amino acrylic acid, hydroxyl acrylic acid, polyurethane acrylate, polycyanoacrylate, polyacrylate, ethylene/acrylic acid copolymers and salts thereof (ionomers), poly(vinyl alcohol), cellulose triacetate, polycarbonate, polyethylene naphthalate, polyethylene terephthalate, polyvinyl alcohol, polymethyl methacrylate, polycyclopentene, aliphatic isocyanate, polymethyl methacrylate, fluorine/silicon-modified polyacrylic acid, and polyisocyanate/polythioether.

9. A polymer material of claim 8, that when tested by a Xenon lamp test has an adhesive force of Grade 1 after 14 days.

10. A polymer material of claim 8, that when tested by a Xenon lamp test has an adhesive force of Grade 1 after 21 days

11. A polymer material of claim 8, that exhibits a Gardner color scale of about 1 to about 8.

12. A polymer material of claim 8, that exhibits a Gardner color scale of about 1 to about 8 with the compound of formula (I) or formula (II) and the polymer material exhibits a Gardner color scale of about 1 to about 8 without the compound of formula (I) or formula (II).

13. An article of manufacture comprising the compound according to claim 1.

14. The article of manufacture according to claim 13, wherein the article of manufacture is selected from an epoxy resin-based article of manufacture and composite articles of manufacture thereof, a polyethylene naphthalate-based article of manufacture, an optical adhesive, an optical element, an optical film, an optical lens, an anti-blue light article of manufacture, a reflective sheet and a conformable marking sheet, a vehicle window and a film thereof, a vehicle paint protection film, an automotive paint, a solar control film, a solar reflector, a reflective printed label, a UV absorbing glass and a glass coating material, an electrochromic device, a film/a glazing, a windshield, or a middle layer.

15. The article of manufacture according to claim 13, wherein the article of manufacture is selected from an epoxy-reinforced carbon fiber composite material comprising an epoxy-reinforced carbon fiber and an outer layer coating thereof; the coating comprises a resin coating material selected from one or more of an amino acrylic resin coating material, a hydroxyl acrylic resin coating material, a polyester resin coating material, a polyurethane acrylate coating material, a polyurethane resin coating material, an epoxy-modified resin coating material, and a polysiloxane-modified resin coating material.

16. The article of manufacture according to claim 13, wherein the article of manufacture is selected from an optical film or an optical element comprising a coating material resin substrate selected from at least one of cellulose triacetate, polycarbonate, polyacrylate, polyethylene naphthalate, polyethylene terephthalate, polyvinyl alcohol, polymethyl methacrylate, polycyclopentene, polyimide, polystyrene and derivatives thereof, an epoxy resin, polyurethane, and polysilane

17. The article of manufacture according to claim 13, wherein the article of manufacture is selected from an anti-blue light lens comprising a resin substrate selected from at least one of aliphatic isocyanate, polymethyl methacrylate, polycarbonate, polyamide, polymethyl methacrylate, fluorine/silicon-modified polyacrylic acid, and polyisocyanate/polythioether.

18. The article of manufacture according to claim 13, wherein the article of manufacture is selected from an automotive paint comprising a cathodic electrodeposition primer and a coating compounded therewith; the electrodeposition primer or the coating compounded therewith comprises a resin coating material; the cathodic electrodeposition primer coating takes an epoxy resin as a matrix, and the cathodic electrodeposition primer coating further comprises a polyester resin, a polyurethane resin, an epoxy/polyester hybrid resin, an acrylic resin, a polysiloxane resin, an amino acrylic resin, a hydroxyl acrylic resin, a polysiloxane-modified resin, and an epoxy-modified resin.

19. A method of providing ultraviolet absorption comprising incorporating a compound according to claim 1 into a composition, polymer material or article of manufacture.

20. The method of claim 19, that provides ultraviolet absorption from 280 nm to 430 nm.
